(19) United States
(12) Patent Application Publication
(10) Pub. No.: US 2018/0353486 A1
(43) Pub. Date: Dec. 13, 2018

(54) METHODS AND PHARMACEUTICAL COMPOSITIONS FOR THE TREATMENT OF DARIER DISEASE

(71) Applicants: INSERM (Institut National de la Sante et de la Recherche Medicale), Paris (FR); Assistance Publique-Hopitaux de Paris (APHP), Paris (JP); Universite Paris Descartes, Paris (FR); Fondation Imagine, Paris Cedex 15 (FR)

(72) Inventors: Alain Hovnanian, Paris (FR); Marina Simon, Paris Cedex 15 (FR)

(21) Appl. No.: 15/780,722

(22) PCT Filed: Nov. 30, 2016
(86) PCT No.: PCT/EP2016/079331

§ 371 Date: Jun. 1, 2018

(30) Foreign Application Priority Data
Dec. 1, 2015 (EP) 15306901.8

(51) Int. Cl. A61K 31/436; A61K 38/13; A61P 17/12

(52) U.S. Cl. CPC A61K 31/436; A61P 17/12; A61K 38/13

(57) ABSTRACT

The present invention relates to methods and pharmaceutical composition for the treatment of Darier disease. In particular, the present invention relates to a method of treating Darier disease in a subject in need thereof comprising administering the subject with a therapeutically effective amount of a calcineurin inhibitor.

### FIELD OF THE INVENTION

The present invention relates to methods and pharmaceutical composition for the treatment of Darier disease.

### BACKGROUND OF THE INVENTION:

Changes in extracellular calcium concentrations which are observed in normal epidermis or after epidermal permeability perturbation, control epidermal functions such as differentiation, barrier repair, keratinocyte cell-to-cell adhesion and keratinocyte motility (Mao-Qiang et al. 1997; Fang et al. 1998; Vasioukhin et al. 2000). Thus, calcium-dependent signal transduction pathways that control these processes are essential for keratinocyte and epidermal viability. In normal epidermis, there is an increasing gradient of extracellular Ca²⁺, from the basal layer to the superficial layers. High extracellular concentrations are required for epidermal intercellular adhesion, differentiation, and cornification. Ca²⁺ signalling involves the binding of raised extracellular Ca²⁺ to a Ca²⁺ plasma membrane receptor, which activates phospholipase C and generates inositol 1,4,5-tri-phosphate (IP3) and diacylglycerol from phosphatidylinositol bisphosphate. IP3 in turn acts as a second messenger, binds to its receptors on the ER to trigger the release of Ca²⁺ from the intracellular stores into the cytoplasm. Emptying of ER Ca²⁺ stores activates an influx of extracellular Ca²⁺ through plasma membrane Ca²⁺ channels, which function like store-operated channels. The increase of intracellular Ca²⁺ levels activates calmodulin, a major Ca²⁺-binding protein. This results in the activation of the cytosolic calmodulin-dependent phosphatase calcineurin, which dephosphorylates NFAT, a major Ca²⁺-dependent transcription factor, allowing its translocation into the nucleus and the induction of downstream target genes (Reviewed by Muller & Rao 2010). Other transcription factors such as CREB and NFkB are also activated by Ca2+ signalling (Sheng et al. 1990; Tabary et al. 2006).

Darier disease (DD) is an acantholytic genetic skin disorder inherited in a dominant manner and characterized by loss of cell-to-cell adhesion and abnormal keratinization (Burge & Hovnanian 2011). DD is caused by mutations in ATP2A2 encoding the sarco/endoplasmic reticulum Ca2+-ATPase isoform 2 (SERCA2) pump which plays a key role in Ca2+ signalling by refilling the internal Ca2+ reservoir of the ER (Sakuntabhai et al. 1999) (OMIM*108740). Functional studies of ATP2A2 mutations identified in DD have shown that Ca²⁺ transport by mutated SERCA2 is abolished in patient keratinocytes (Dode et al. 2003; Miyauchi et al. 2006), leading to depleted ER Ca²⁺ stores (Foggia et al. 2006; Pani et al. 2006) (Hovnanian 2004; Savignac et al. 2011). However, the consequences of this depletion of Ca²⁺ stores remain controversal. Leinonen et al. have reported that intracytoplasmic Ca²⁺ ([Ca^{2+]}) is increased in Darier keratinocytes (Leinonen et al. 2005), whereas Foggia et al. have shown a decrease in [Ca²]ᵢ, as a result of upregulation of ATP2C1, encoding the human secretory pathway Ca²⁺/Mn²⁺-ATPase (hSPCA1), i.e. the Ca²⁺ pump of the Golgi apparatus (Foggia et al. 2006). To date, no animal model is available for DD: atp2a2 knockout is lethal in mice and aged atp2a2^{+/-} mice develop squamous cell carcinomas but no DD like lesions (Liu et al. 2001). For these reasons, Darier keratinocytes represent a unique opportunity to explore the mechanisms underlying DD physiopathology.

Calcineurin inhibitors such as systemic Cyclosporin and topical Tacrolimus have been successfully used in few isolated cases to treat Darier disease when retinoids were not recommended (Rubegni et al. 2006; Pérez-Carmona et al. n.d.; Stewart & Yell 2008; Shahidullah et al. 1994). The therapeutic effects of calcineurin inhibitors in eczema and inflammatory skin diseases have been previously attributed to their well-documented effects on T cells.

### SUMMARY OF THE INVENTION

The present invention relates to methods and pharmaceutical composition for the treatment of Darier disease. In particular, the present invention is defined by the claims.

### DETAILED DESCRIPTION OF THE INVENTION

Keratinocyte differentiation, adhesion and motility are directed by extracellular Ca²⁺ concentration increases, which in turn enhance intracellular Ca²⁺ levels. Normal keratinocytes, in contrast to most non-excitable cells, require Ca²⁺ release from both Golgi and endoplasmic reticulum Ca²⁺ stores for efficient Ca²⁺ signaling. Darier disease is a genetic skin disease caused by loss of function in ATP2A2 encoding the endoplasmic reticulum ATPase SERCA2. Dysfunction of this pump impairs calcium homeostasis which is likely to modify the response to external calcium signal and transcription of Ca²⁺-dependent genes. To address this question, the inventors compared microarray analysis from normal (NKs) and Darier keratinocytes (DKs). They confirm that DKs display intrinsic premature differentiation and they identify differentially expressed genes from the “epidermal differentiation complex”.

The inventors show that NFATc1, a key calcium dependent transcription factor, is up-regulated and activated in DKs in basal conditions and is involved in premature signature differentiation in DKs. Finally, NFATc1 inhibition using calcineurin inhibitors or NFATc1-siRNA decreased the expression of differentiation genes in DKs, providing mechanistic bases to support the clinical use of calcineurin inhibitor in Darier disease.

Accordingly, a first object of the present invention relates to a method of treating Darier disease in a subject in need thereof comprising administering the subject with a therapeutically effective amount of a calcineurin inhibitor.

As used herein, the term “Darier disease” has its general meaning in the art. Darier disease (DD) is a keratinization disorder characterized by the development of keratotic papules in seborrheic areas and specific nail anomalies. The prevalence is estimated at around 1/50,000. Onset of the disease usually occurs around puberty. Patients present with greasy and colored (yellow-brown or brown) keratotic papules, which may be isolated or grouped forming plaques. The skin lesions often become infected and malodorous, and are responsible for major discomfort. They may be exacerbated by exposure to sunlight or artificial UVB radiation, heat, sweating, friction, and infections. The sites of predilection are the seborrheic areas of the trunk and face: upper chest, back, sides of the neck, forehead, ears, and scalp. The flexures are also frequently involved (the groins, axillae, and anogenital region). Hands and feet may also show discrete papules on the dorsal surfaces. Careful examination of the palms and soles frequently reveals small pits or punctuated keratoses that are highly suggestive, if not specific, of DD. Nail abnormalities are almost constant and highly suggestive. The nails show the specific combination of red and white longitudinal stripes and present subungual hyperkeratosis. They are fragile and have a V-shaped defect. The hard palate, oral mucosa, esophagus, vulva and rectum may be the site of whitish small papules, often densely grouped (leukoplakia). Patients have an increased susceptibility to herpes simplex and pyogenic infections. Severity of the disease is highly variable, even within the same family.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In particular, the method of the present invention is particularly suitable for preventing the development of Darier lesions (e.g. preventing the apparition of new lesions) by restoring skin differentiation.

As used herein, the term “calcineurin inhibitor” has its general meaning in the art and refers to substances which block calcineurin (i.e. calcium/calmodulin-regulated protein phosphatase involved in intracellular signalling) dephosphorylation of appropriate substrates, by targeting calcineurin phosphatase (PP2B, PP3), a cellular enzyme that is involved in gene regulation. A calcineurin inhibitor of the present invention is typically an immunophilin-binding compound having calcineurin inhibitory activity. Immunophilin-binding calcineurin inhibitors are compounds forming calcineurin inhibiting complexes with immunophilins, e.g. cyclophilin and macrophilin. Examples of cyclophilin-binding calcineurin inhibitors are cyclosporines or cyclosporine derivatives (hereinafter cyclosporines) and examples of macrophilin-binding calcineurin inhibitors are ascomycin (FR 520) and ascomycin derivatives (hereinafter ascomycins). A wide range of ascomycin derivatives are known, which are either naturally occurring among fungal species or are obtainable by manipulation of fermentation procedures or by chemical derivatization. Ascomycin-type macrolides include ascomycin, tacrolimus (FK506), sirolimus and pimecrolimus. Cyclosporine, originally extracted from the soil fungus Potypaciadium infilatum, has a cyclic 11-amino acid structure and includes e.g. Cyclosporines A through I, such as Cyclosporine A, B, C, D and G. Voclosporin is a next-generation calcineurin inhibitor that is a more potent and less toxic semi-synthetic derivative of cyclosporine A. In some embodiments, the calcineurin inhibitor of the present invention is the trans-version of voclosporin, trans-ISA247 (Cas number 368455-04-3) which is described in, for example, US Patent Publication No.: 2006/0217309, which is hereby incorporated herein by reference. Further compositions of voclosporin are described, for example, in U.S. Pat. No. 7,060,672, which is hereby incorporated herein by reference. Tacrolimus (FK506) is another calcineurin inhibitor which is also a fungal product, but has a macrolide lactone structure. Sirolimus (rapamycin) is a microbial product isolated from the actinomycete Streptomyces hygroscopicus. Sirolimus binds to an immunophilin (FK-binding protein 12, FKBP12) forming a complex, which inhibits the mammalian target of rapamycin (mTOR) pathway through directly binding the mTOR Complexi (mTORC1). Pimecrolimus is also a calcineurin inhibitor. Calcineurin inhibitors such as cyclosporine A, voclosporin, ascomycin, tacrolimus, pimecrolimus, an analog thereof, or a pharmaceutically acceptable salt thereof, can be utilized in a mixed micellar composition of the present disclosure.

By a “therapeutically effective amount” is meant a sufficient amount of the calcineurin inhibitor to treat Darier disease at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the agent for the symptomatic adjustment of the dosage to the subject to be treated. A medicament typically contains from about 0.01 mg to about 500 mg of the agent, preferably from 1 mg to about 100 mg of the agent. An effective amount of the drug is ordinarily supplied at a dosage level from 0.0002 mg/kg to about 20 mg/kg of body weight per day, especially from about 0.001 mg/kg to 7 mg/kg of body weight per day.

Typically the calcineurin inhibitor of the present invention is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. In some embodiments, it may be desirable to administer the calcineurin inhibitor of the present in a topical manner (e.g. application to the subject's skin). In some embodiments, the calcineurin inhibitor is administered on non-lesional skin of the subject. The topical pharmaceutically acceptable carrier is any substantially nontoxic carrier conventionally usable for topical administration of pharmaceuticals in which the calcineurin inhibitor of the present invention will remain stable and bioavailable when applied directly to skin surfaces. For example, carriers such as those known in the art effective for penetrating the keratin layer of the skin into the stratum comeum may be useful in delivering the calcineurin inhibitor of the present invention to the area of interest. Such carriers include liposomes. calcineurin inhibitor of the present invention can be dispersed or emulsified in a medium in a conventional manner to form a liquid preparation or mixed with a semi-solid (gel) or solid carrier to form a paste, powder, ointment, cream, lotion or the like. Suitable topical pharmaceutically acceptable carriers include water, buffered saline, petroleum jelly (vaseline), petrolatum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, synthetic polymers, alcohols, polyols, and the like. The carrier can be a water miscible carrier composition. Such water miscible, topical pharmaceutically acceptable carrier composition can include those made with one or more appropriate ingredients outset of therapy. Because dermatologic conditions to be treated may be visible, the topical carrier can also be a topical acceptable carrier. The topical acceptable carrier will be any substantially non-toxic carrier conventionally usable for topical administration in which calcineurin inhibitor of the present invention will remain stable and bioavailable when applied directly to the skin surface. Suitable cosmetically acceptable carriers are known to those of skill in the art and include, but are not limited to, cosmetically acceptable liquids, creams, oils, lotions, ointments, gels, or solids, such as conventional cosmetic night creams, foundation creams, suntan lotions, sunscreens, hand lotions, make-up and make-up bases, masks and the like. Any suitable carrier or vehicle effective for topical administration to a patient as know in the art may be used, such as, for example, a cream base, creams, liniments, gels, lotions, ointments, foams, solutions, suspensions, emulsions, pastes, aqueous mixtures, sprays, aerosolized mixtures, oils such as Crisco®, soft-soap, as well as any other preparation that is pharmaceutically suitable for topical administration on human and/or animal body surfaces such as skin or mucous membranes. Topical acceptable carriers may be similar or identical in nature to the above described topical pharmaceutically acceptable carriers. It may be desirable to have a delivery system that controls the release of calcineurin inhibitor of the present invention to the skin and adheres to or maintains itself on the skin for an extended period of time to increase the contact time of the calcineurin inhibitor of the present invention on the skin. Sustained or delayed release of calcineurin inhibitor of the present invention provides a more efficient administration resulting in less frequent and/or decreased dosage of calcineurin inhibitor of the present invention and better patient compliance. Examples of suitable carriers for sustained or delayed release in a moist environment include gelatin, gum arabic, xanthane polymers. Pharmaceutical carriers capable of releasing the calcineurin inhibitor of the present invention when exposed to any oily, fatty, waxy, or moist environment on the area being treated, include thermoplastic or flexible thermoset resin or elastomer including thermoplastic resins such as polyvinyl halides, polyvinyl esters, polyvinylidene halides and halogenated polyolefins, elastomers such as brasiliensis, polydienes, and halogenated natural and synthetic rubbers, and flexible thermoset resins such as polyurethanes, epoxy resins and the like. Controlled delivery systems are described, for example, in U.S. Pat. No. 5,427,778 which provides gel formulations and viscous solutions for delivery of the calcineurin inhibitor of the present invention to a skin site. Gels have the advantages of having a high water content to keep the skin moist, the ability to absorb skin exudate, easy application and easy removal by washing. Preferably, the sustained or delayed release carrier is a gel, liposome, microsponge or microsphere. The calcineurin inhibitor of the present invention can also be administered in combination with other pharmaceutically effective agents including, but not limited to, antibiotics, other skin healing agents, and antioxidants. In particular, topical calcineurin inhibitors, such as a tacrolimus ointment, which is commercially available as PROTORIC®, have been used to treat atopic dermatitis, which is an eczematous skin disease that has typically been treated with topical steroids. A tacrolimus ointment, which is commercially available as PROTOPIC®, has been reported to inhibit calcineurin. Another commercially available calcineurin inhibitor is pimecrotimus, which is commercially available in a cream and sold as in a cream form, commercially ELIDEL®.

A second object of the present invention relates to a method of treating Darier disease in a subject in need thereof comprising administering the subject with a therapeutically effective amount of an inhibitor of NFATC1 gene expression.

As used herein, the term ‘NFATc1” has its general meaning in the art and refers to Nuclear factor of activated T-cells, cytoplasmic 1 is a protein that in humans is encoded by the NFATC1 gene (Rao A, Luo C, Hogan P G (1997). “Transcription factors of the NFAT family: regulation and function”. Annu. Rev. Immunol. 15: 707-47. doi:10.1146/annurev.immunol.15.1.707). An exemplary human nucleic acid sequence is represented by the NCBI Reference Sequence: NM_001278669.1. An exemplary amino acid sequence is represented by NCBI Reference Sequence: NP_001265598.1.

In another embodiment, the inhibitor according to the invention is an inhibitor of gene expression. An “inhibitor of gene expression” refers to a natural or synthetic compound that has a biological effect to inhibit or significantly reduce the expression of a gene. Inhibitors of gene expression for use in the present invention may be based on anti-sense oligonucleotide constructs. Anti-sense oligonucleotides, including anti-sense RNA molecules and anti-sense DNA molecules, would act to directly block the translation of the mRNA by binding thereto and thus preventing protein translation or increasing mRNA degradation, thus decreasing the level of the protein (e.g. NFATC1), and thus activity, in a cell. For example, antisense oligonucleotides of at least about 15 bases and complementary to unique regions of the mRNA transcript sequence encoding the targeted protein (e.g. NFATC1) can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of gene expression for use in the present invention. Gene expression can be reduced by contacting a subject or cell with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). Ribozymes can also function as inhibitors of gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays. Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone. Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and in particular cells expressing the targeted proteins (e.g. NFATC1). In particular, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art. Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991). Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion. Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

The invention will be further illustrated by the following figures and examples. However, these examples and figures should not be interpreted in any way as limiting the scope of the present invention.

### FIGURES

FIG. 1: Comparative study in DKs versus NKs. a. The top 15 up-regulated genes in DK versus NK, with a fold induction ratio ≥1.2 and a p-value ≥0.05. Microarrays analysis was performed on human keratinocytes from four healthy donors and five Darier patients.

FIG. 2. Validation of microarrays data. a. QRT-PCR confirmation of several of the differentially expressed genes. The results are shown as fold induction and relative to the expression of the housekeeping gene PGK and indicate the mean+/−SEM n=5. b. QRT-PCR experiments of loricrin, involucrin (IVL) and fillagrin (FLG) expression in DK compared to NK. c. Western Blot validation of increased expression of NFATc1, LCE, SPRR and loricrin proteins. Actin was used for normalization. *P<0.05 and ***P<0.001.

FIG. 3. a. NFATc1 expression and nuclear localization is increased in DKs compared to NKs. Reversion by calcineurin inhibitors Normal (NK) and Darier (D2, D3, D4) keratinocytes were grown in 0.06 mM Ca2+ (a) and cells are treated or not with 10 μM cyclosporin A (b) or 100 ng/ml tacrolimus (c) for 24 hours. Nuclear (N) and cytoplasmic (C) cell extracts were immunoblotted for NFATc1 and actin for normalization.

FIG. 4: TG-treated NK display the same features as DK. NKs were pretreated (TG) or not (−) with 1 μM of TG for 24 hours. a-b. Thapsigargin induces increased NFATc1 expression and nuclear localization in TG-treated NK. Reversion by CsA treatment. c-d. TG-treated NK display premature differentiation markers up-regulation. SPRR2, LCE3B-E and loricrin expression is induced after 24 h (TG24) and 48 h (TG48) of TG-treatment in NK. *P<0.05. Results are from NKs from two donors (a) and one donor (b-d) and are representative of two independent experiments from two NKs.

FIG. 5: Calcineurin inhibitors and NFATc1 inhibition decreased premature differentiation in DK. Quantitative RT-PCR and western blot were performed on mRNA and protein extracts from five DKs after 48 h Tacrolimus (a-b) or 24 h and 48 h CsA treatment (b). For (a), results are expressed as fold repression compared to non-treated DK and are the mean+/−SEM of five DK. Results are representative of two independent experiments. Asterisks represent statistical significance versus the appropriate control in each case. *P<0.05 and***P<0.001 c. Relative efficiency of small interfering RNA (siRNA)-mediated NFATc1 silencing compared to scrambled control by quantitative PCR and western blotting. Decreased expression of differentiation genes after NFATc1 specific inhibition at mRNA (d). For (d), results are expressed as fold repression compared to DKs transfected with scrambled si-RNA and are the mean+/−SEM of four DK from one experiment.

### EXAMPLE

Results

Microarrays Analysis: DKs Display Premature Differentiation

To identify new genes likely to be involved in the physiopathological cascade of DD, we compared gene expression profile of normal (NKs) and DKs caning out a microarray analysis.

Keratinocytes from four healthy donors and five Darier patients were grown in proliferating conditions and total RNA was extracted.

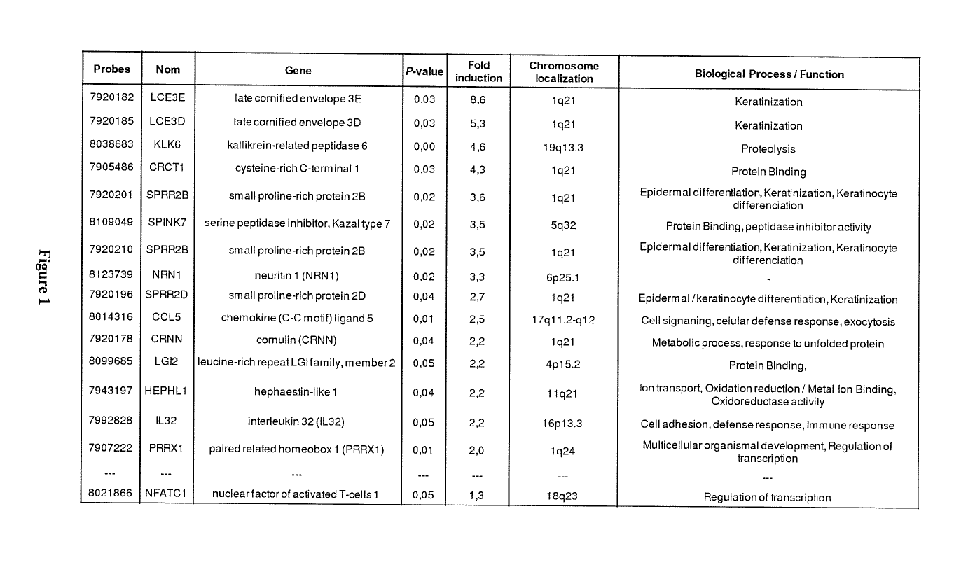

Seven out of fifteen of the most upregulated genes in DK belong to biological processes involved in epidermal differentiation and map to the same chromosomal region: 1q2 1 known as the epidermal differentiation complex (EDC) (FIG. 1) (Mischke et al. 1996; Marenholz et al. 2001). The EDC is a 2-megabase region containing multiple families of genes encoding structural and regulatory proteins involved in cornified envelope formation of the epidermis and in the regulation of keratinocyte terminal differentiation. Four main groups are part of the EDC, known as small prolin rich proteins (SPRR), Late cornified envelope genes (LCE), S100 and S100-fused type proteins.

In our study, LCE3D and LCE3E were strongly upregulated in DK with a 8.6 and 5.3 fold induction respectively compared to normal keratinocytes (NKs). LCE genes are expressed late during keratinocyte differentiation in the granular keratinocytes (Henry et al. 2012). They are among the latest cornified envelope components to be cross-linked to the structure. Two isoforms of the Small Prolin Rich Proteins (SPRR2B and 2D) displayed a 3.6 and 2.7 fold increase respectively. SPRR proteins are precursors of the cornified envelope and are critical for skin barrier function (Henry et al. 2012). These proteins are mainly cross-linked to loricrin in the cornified envelope. Two other EDC genes, Cornulin and cystin-rich C-terminal 1 were also overexpressed in DK.

In contrast, the most commonly known genes involved in keratinocytes differentiation such as loricrin, filaggrin and involucrin were not statistically differentially expressed in DK compared to NK, although loricrin reached significance in 3 of 5 patients.

These observations indicated that Darier keratinocytes display premature differentiation features in basal culture conditions.

Transcriptions factors. To explain upregulation of genes involved in keratinocyte differentiation in DK, we searched for some differentially expressed transcription factors that could modulate their expression. Two candidates genes were identified, NFATc1 and Elf3 displaying a 1.2 and a 1.93 fold increase respectively in DK compared to NK.

Nuclear factor activated T cells (NFATc) proteins consist in a family of transcription factors whose activation is controlled by calcineurin, a calcium-dependant phosphatase. Four distinct genes encoding closely related NFATc proteins (NFATc1-c4) (Yang et al. 2002) have been identified and are involved in multiple biological processes ranging from lymphocyte activation to the development of cardiac hypertrophy (Molkentin et al. 1998). Moreover, NFAT was also shown to be involved in the differentiation processes of different cell type such as adipocytes (Ho et al. 1998), muscular cells (Delling et al. 2000), neuregulin-regulated Scwann cells (Kao et al. 2009), intestinal cells (Wang et al. 2001), as well as in primary keratinocyte differentiation (Santini et al. 2001).

The E26 transformation-specific (ETS) family of transcription factors is composed of 27 members in human which are known to regulate many different biological processes, including cell proliferation, cell differentiation, embryonic development and inflammation. Expression of the epithelium-specific ETS transcription factor-1 (ESE-1 or Elf3) was increased in DK versus NK in our microarray analysis. This factor has previously been shown to play a role in the regulation of keratinocyte differentiation markers expression such as SPRR1B (Reddy et al. 2003) in human keratinocytes and could contribute to regulate expression of EDC genes.

Validation of Microarrays Observations: NFATc1 and EDC Genes Expression is increased in DKs.

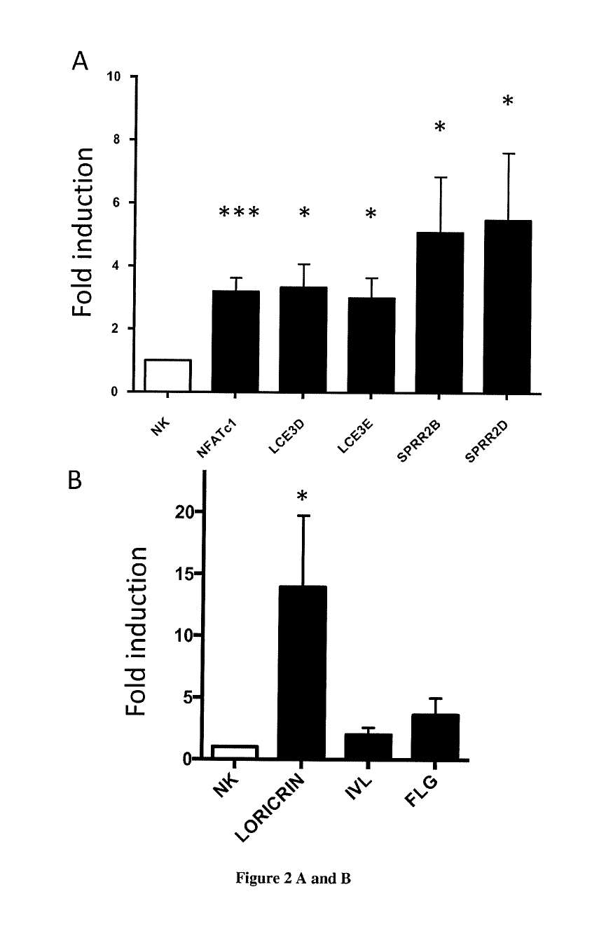

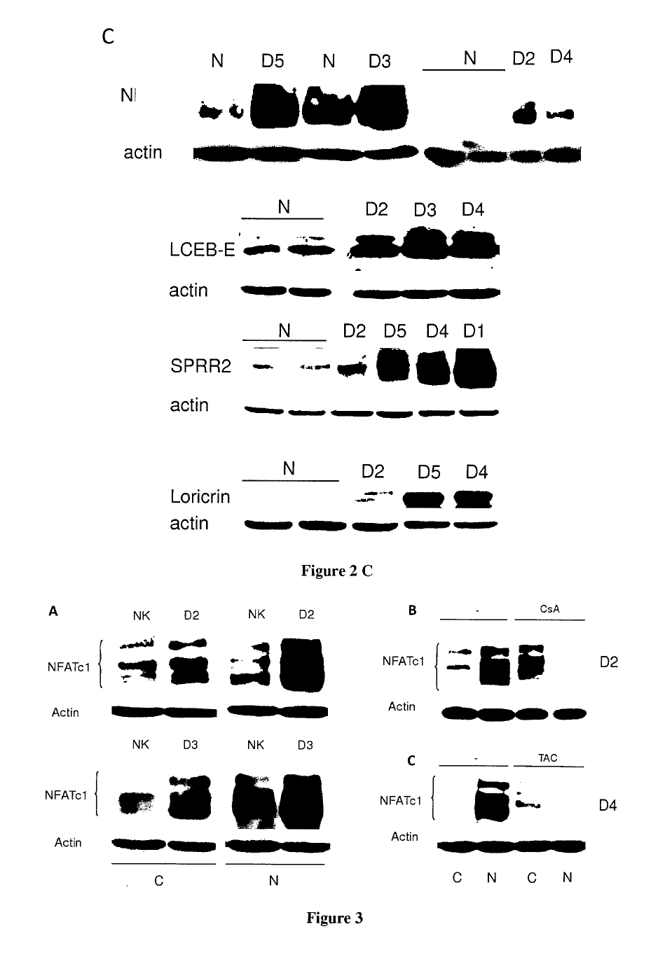

In order to confirm increased expression of NFATc1, Elf3 and EDC genes in DKs, quantitative RT-PCR was performed on total cell extracts from healthy and DKs grown in low calcium medium. Quantitative RT-PCR revealed that NFATc1 transcripts were over-expressed in all five DKs tested as compared to NKs (about 3 fold increase) (FIG. 2a) in contrast to Elf3 which remained comparable between normal and DKs (data not shown). NFATc1 increase was further confirmed by western blot analysis on total cell lysates from DK maintained in proliferating conditions using a specific antibody directed against the NFATc1 isoform (FIG. 2c).

Similar experiments confirmed upregulation of genes from the EDC including LCE3D, LCE3E, SPRR2B, SPRR2D previously identified by microarray analysis. Quantitative RT-PCR (FIG. 2a) and western blot (FIG. 2c) analyses confirmed increased expression at the mRNA and protein level of these EDC proteins for the five patients studied. Moreover, we controlled the expression levels of the genes most commonly involved in the keratinocyte differentiation process, which were not statistically modified in the microarrays experiment. Quantitative RT-PCR experiments revealed that loricrin expression was statistically increased in DK compared to NK, but involucrin and fillagrin expression was not statistically modified, confirming microarray data (FIG. 2b).

NFATc1 is Overexpressed and Activated in DKs.

NFATc1, which exists in a highly phosphorylated and inactive form in the cytoplasm, translocates into the nucleus upon its dephosphorylation by the phosphatase calcineurin in response to an increase in intracellular calcium. Once in the nucleus, NFATc1 binds to enhancer elements of specific genes leading to their transcriptional activation (Hogan et al. 2003). Here, we investigated NFATc1 localization using nuclear and cytoplasmic extracts from NKs and DKs grown in basal conditions and could demonstrate that a large amount of NFATc1 protein was located in the nucleus of DK compared to NK (FIG. 3a). These results point to the activation of the calcineurin pathway in DKs.

NFATc1 Nuclear Localization in DKs can be Reversed by Calcineurin Inhibitors: Cyclosporin A and Tacrolimus.

CsA and Tacrolimus are well characterized calcineurin inhibitors which bind and suppress calcineurin activity acting as a complex with cyclophilins and FK-binding protein respectively. (Schreber 1991; Siekierka & Sigal 1992). In a variety of cells, this leads to an increased phosphorylation of calcineurin substrates, particularly NFAT, preventing NFAT translocation to the nucleus. Therefore, we treated healthy keratinocytes and DKs with 10 μM of CsA or 100 ng/ml of Tacrolimus for 24 hours. Western blot experiments using a specific antibody directed against NFATc1 were performed on nuclear and cytosolic extracts. In these conditions, we showed that cyclosporin A (FIG. 3b) and Tacrolimus (FIG. 3c) treatments induced NFATc1 translocation from the nucleus to the cytoplasm in DKs, suggesting that NFATc1 nuclear localization in DKs is calcineurin-dependant.

TG Induces Premature Differentiation and NFATc1 Increase in NK

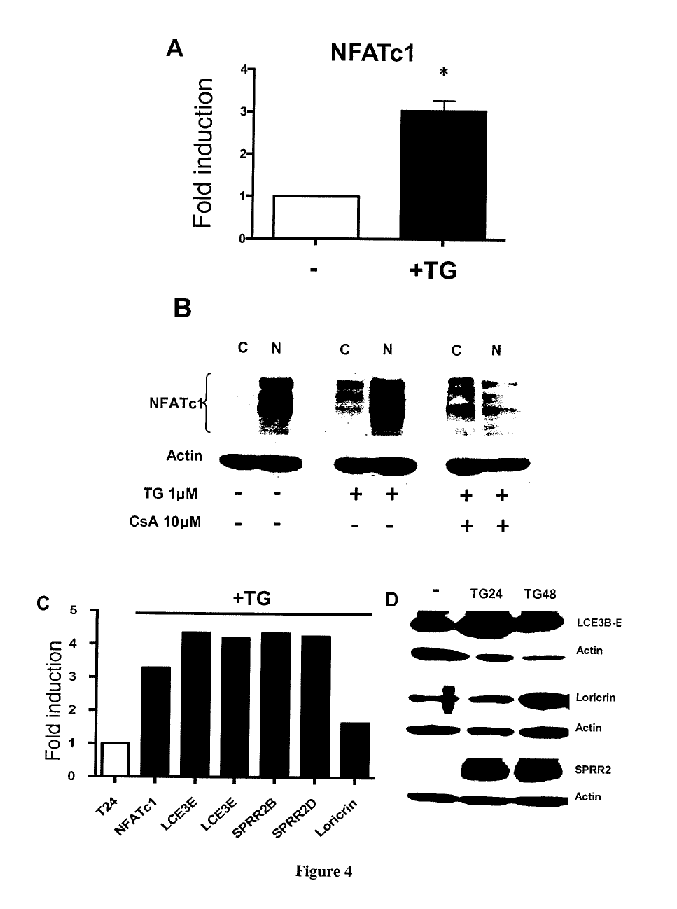

To test whether loss of SERCA2 function is sufficient to induce NFATc1 overexpression and activation associated with premature expression of EDC genes, NKs were treated with 1 μM of thapsigargin (TG), a SERCA pumps inhibitor, for 24 and/or 48 hours to mimick SERCA2 dysfunction in DK. Quantitative RT-PCR revealed increased NFATc1 expression in TG-treated cells (FIG. 4a). Western blot analysis of total protein extracts (FIG. 4b) was performed to test whether NFATc1 was in its active form in TG-treated NK, nuclear extracts were prepared and western blot analysis revealed that a higher amount of NFATc1 protein was located in the nucleus of TG-treated NKs compared to non-treated NKs. These results indicated that NFATc1 dephosphorylation and activation is enhanced after treatment with a SERCA inhibitor. TG-treated NKs were subjected to 10 μM CsA which induced NFATc1 translocation from the nucleus to the cytoplasm (FIG. 4b), as previously observed in DKs (FIG. 3b). These results demonstrate that increased NFATc1 expression and its nuclear abundance in DKs are consequences of loss of SERCA2 function, highlighting for the first time the involvement of the calcineurin-dependant pathway in DD pathophysiology.

In order to confirm that upregulation of differentiation genes is due to SERCA2 defect, qRT-PCR and western blot analysis were performed on TG-treated NKs.

Quantitative RT-PCR and western blot analysis using specific antibodies against LCE3B-E and SPRR2 proteins from total cell extracts revealed increased expression of LCE3D, LCE3E, SPRR2B, SPRR2D and loricrin transcripts and proteins in TG-treated NKs (FIG. 4c-d).

NFATc1 Invalidation Reverses Premature Differentiation in DKs.

Having confirmed that NFATc1 and EDC gene expression is increased in DK in basal conditions, we further investigated the role of NFATc1 in regulating premature differentiation observed in DKs. Indeed, previous studies in keratinocytes have shown that NFAT mediated transcription is activated during keratinocyte differentiation and that NFAT activates the expression of terminal differentiation markers such as loricrin, profilaggrin, keratin 1, as well as cell cycle inhibitors such as p21 and p27 (Santini et al. 2001; Mammucari et al. 2005).

To substantiate the role of NFATc1 as an enhancer of EDC genes expression in DKs and TG-treated NKs grown in basal condition, we inhibited NFATc1 activation indirectly (using calcineurin inhibitors) and directly (using small interfering RNA-mediated NFATc1 silencing), in Darier keratinocytes.

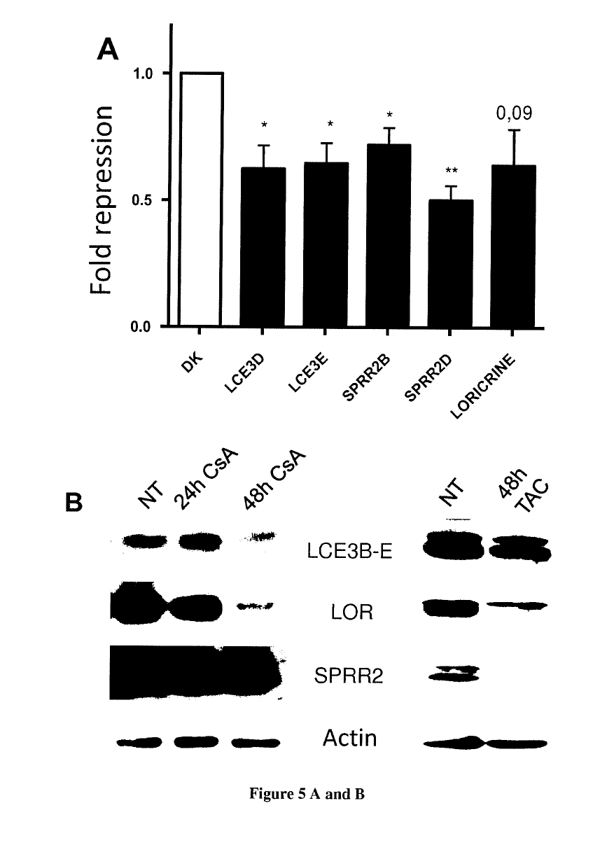

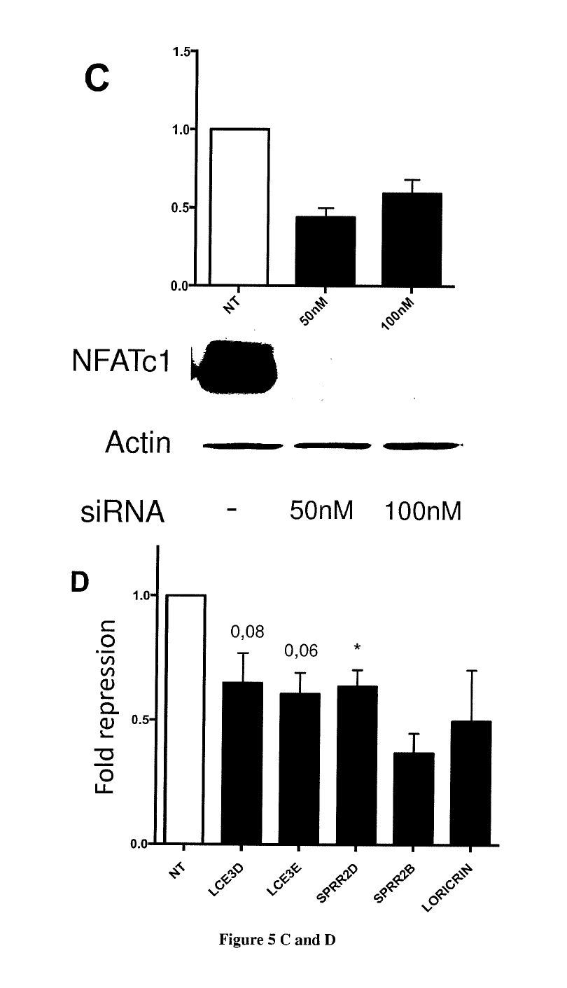

DKs maintained in proliferating conditions were treated with 100 ng ml⁻¹ of Tacrolimus for 24 hours. Quantitative RT-PCR on total cell extracts showed that expression of LCE3D, LCE3E, SPRR2B, SPRR2D and loricrin was significantly decreased in Tacrolimus-treated DKs (FIG. 5a). Similar results were obtained after 10 μmol L⁻¹ Cyclosporin treatment (data not shown). Western blot experiments confirmed these results at the protein level by showing reduced LCE3B-E, SPRR2 and loricrin expression after CsA and Tacrolimus treatment (FIG. 5b). siRNA-mediated knock-down of NFATc1, the efficiency of which was verified by quantitative RT-PCR and western blotting (FIG. 5c), significantly decreased EDC gene expression, at the mRNA and protein levels, when compared to cells transfected with non-targeting control siRNA.(FIG. 5d).

Taken together, these results demonstrate that NFAT and more especially the NFATc1 iso form and the calcineurin/calmodulin pathway are involved in premature differentiation observed in DKs, and are likely to contribute to abnormal keratinization, a clinical feature of the skin Darier patients.

Discussion:

This study demonstrates that NFATc1, a major calcium-dependent transcription factor, is overexpressed and activated in DKs. We show that several genes of the EDC are upregulated in DKs (LCE3D, LCE3E, SPRR2B, SPRR2D and loricrin). NFATc1 plays a key role in premature expression of these EDC genes. NFATc1 inhibition using calcineurin inhibitors or specific siRNA delays premature expression of differentiation genes from the EDC.

Histopathological features of DD include suprabasal acantholysis, hyperkeratosis (thickening of the stratum corneum) and dyskeratosis (abnormal differentiation of single cells so-called corps ronds and grains of Darier).

Here, we show that DKs from perilesional DD skin display premature expression of differentiation markers such as LCE3D-3E, SPRR2B-2D and loricrin. All these genes are part of the epidermal differentiation complex (EDC) located on chromosome 1q21 containing many genes associated with epidermal terminal differentiation. LCE genes respond to environmental stimuli such ultraviolet light and also calcium levels (Jackson et al. 2005), SPRR proteins constitute cornified cell envelope precursors which associate with loricrin and are also UV inducible (Kartasova & van de Putte 1988). In pathophysiological conditions, the litterature has repeatedly reported SPRR and LCE expression changes in several keratinization and skin inflammatory disorders (Ishida-Yamamoto et al. 1995) (Koizumi et al. 1996; Hohl et al. 1995; Hoffjan & Stemmler 2007; Molin et al. 2011; Xu et al. 2011). Consistent with these data, we found that expression of 2 LCE and SPRR isoforms was increased in DKs.

Premature differentiation in DD was reported in previous studies, but the mechanisms involved were not elucidated. DD lesional skin was shown to display a major increase in involucrin staining (Kanitakis et al. 1987) with premature and enhanced expression at keratinocyte cell membrane and cytoplasm in the lower epidermal layers (Kassar et al. 2008). Involucrin expression was not statistically modified in our study, which might be due to the fact that we worked on DKs from non lesional skin. All other studies that showed involucrin overexpression and premature differentiation were performed on lesional skins sections, which might induce different pattern of differentiation gene expression.

Our work gives new insights on DD molecular pathogenesis. We show NFATc1 overexpression and increased nuclear localization in DKs compared to NKs. To our knowledge, no study has previously evidenced deregulation of NFATc1 in Darier disease. Our results are consistent with a previous study which showed that SERCA2+/− mice display increased expression of NFATc1 in keratinocytes (Hong et al. 2010).

A possible explanation could be that SERCA2 dysfunction would induce local calcium increase in DKs cytoplasm, thus activating calcineurin. Once triggering, calcineurin could dephosphorylate NFAT, inducing its nuclear translocation and targeted gene transcription.

The expression of other NFAT isoforms was not modified in our study (data not shown). This could be explained by the fact that the smallest isoform of NFATc1 is the only NFAT protein to be subjected to positive autoregulation (Chuvpilo et al. 1999; Zhou et al. 2002; Serfling et al. 2006).

The observation that TG-treated NK recapitulated NFATc1 increased nuclear localization, as well as premature expression of LCE3D, LCE3E, SPRR2B, SPRR2D and Loricrin as observed in DKs indicates that loss of SERCA2 function is sufficient to cause NFATc1 overexpression and activation leads to premature expression of differentiation markers.

NFAT proteins have been involved in the differentiation processes of different cell type such as adipocytes (Ho et al. 1998), muscular cells (Delling et al. 2000), neuregulin-regulated Schwann cells (Kao et al. 2009), intestinal cells (Wang et al. 2001), as well as in primary normal keratinocyte differentiation (Santini et al. 2001). Indeed, Santini et al. evidenced that primary keratinocyte cell differentiation is associated with nuclear localisation of NFATc1 and 2 isoforms. Another study form Pena et al. (2009), suggested that calcineurin Aα activity is required for normal differentiation of epidermal cells (Pena et al. 2010). Finally, the 2A isoform of the SPRR protein carries AP1 fixation sites in its promotor region, AP1 being a well known cofactor of NFAT for enhancing gene transcription (Macian et al. 2001).

These observations led us to hypothesize that overexpression and overactivation of NFATc1 observed in DKs could be involved in premature differentiation of these cells.

In support of this possibility, we show that NFATc1 inhibition using calcineurin inhibitors or specific siRNA molecules decreases of LCE3D, LCE3E, SPRR2B, SPRR2D and Loricrin expression, confirming the role of NFATc1 in premature differentiation observed in DKs and the activation of the calcineurin/calmodulin pathway in DKs.

Recently, Mauro's group has studied the role of the shingolipid signaling pathway in TG-treated and in SERCA2b siRNA-treated keratinocytes. Sphingosine levels were enhanced, shingosine kinase 1 expression was decreased and inhibition of sphingosine lyase reversed abnormal differentiation (Celli et al. 2012). Moreover, in a previous study, we showed that DKs display ER stress (Savignac et al. 2014) and ER stress is known to induce abnormal keratinization (Reviewed in Sugiura 2013). These results reveal pleiomorphic effects of loss of SERCA2 function on epithelial cells homeostasis and further illustrate the complexity of intercrosses between different calcium signalling pathways.

Other studies (Hampton et al. 2012) showed that NFATc1 is involved in keratinocytes proliferation. Proliferation and differentiation are two related processes. NFAT has a relatively low affinity for DNA and combination with other transcription factors, is required to activate promoters (reviewed in Hogan et al. 2003). The involvement of NFATc1 in proliferation or differentiation process could be the result of the nature of co-factors associates with it.

Oral retinoids still remain the first line treatment for generalized or severe Darier disease (Cooper & Burge 2003). However, a majority of patients treated with oral retinoids experience dose-related side-effects including mucosal dryness, nose bleeding, skin fragility and itching (Burge et al. 1981). Calcineurin inhibitors such as systemic Cyclosporin and topical Tacrolimus have been successfully used in few isolated cases to treat Darier disease when retinoids were not recommended (Rubegni et al. 2006; Pérez-Carmona et al. n.d.; Stewart & Yell 2008; Shahidullah et al. 1994). The therapeutic effects of calcineurin inhibitors in eczema and inflammatory skin diseases have been previously attributed to their well-documented effects on T cells. Here, we show that these drugs are exerting a direct effect on keratinocytes, independent of T-cells, which is likely to have important implications for our understanding of DD pathogenesis and future therapeutic developments.

Our results were obtained using DKs isolated from perilesional skins of Darier patients. It would be interesting to test calcineurin inhibitors on non-lesional skin, in order to prevent the development of Darier lesions. Indeed, if skin differentiation is restored, it might be possible that the skin barrier would be more effective and thus prevent the apparition of new lesions

In conclusion, this work identifies NFATc1 as a key player in DD pathogenesis and highlights the alteration of the calcineurin/calmodulin pathway in DKs. Moreover, we propose a mechanistic explanation for premature differentiation observed in DKs. The observation that calcineurin inhibitors are able to restore proper differentiation in vitro provides a new rational for preventive and/or curative therapeutic option and the basis for future clinical evaluation of calcineurin inhibitors in Darier patients.

### REFERENCES

Throughout this application, various references describe the state of the art to which this invention pertains. The disclosures of these references are hereby incorporated by reference into the present disclosure.

Burge, S. & Hovnanian, A., 2011. Acantholytic disorders of the skin. Fitzpatrick's Dermatology in General Medecine 2011, Chapter 51, pp. 550-556.

Burge, S. M. et al., 1981. The efficacy of an aromatic retinoid, Tigason (etretinate), in the treatment of Darier's disease. The British journal of dermatology, 104(6), pp. 675-9.

Celli, A. et al., 2012. SERCA2-controlled Ca²+-dependent keratinocyte adhesion and differentiation is mediated via the sphingolipid pathway: a therapeutic target for Darier's disease. The Journal of investigative dermatology, 132(4), pp. 1188-95.

Chuvpilo, S. et al., 1999. Multiple NF-ATc isoforms with individual transcriptional properties are synthesized in T lymphocytes. Journal of immunology (Baltimore, Md.: 1950), 162(12), pp. 7294-301.

Cooper, S. M. & Burge, S. M., 2003. Darier's disease: epidemiology, pathophysiology, and management. American journal of clinical dermatology, 4(2), pp. 97-105.

Delling, U. et al., 2000. A Calcineurin-NFATc3-Dependent Pathway Regulates Skeletal Muscle Differentiation and Slow Myosin Heavy-Chain Expression A Calcineurin-NFATc3-Dependent Pathway Regulates Skeletal Muscle Differentiation and Slow Myosin Heavy-Chain Expression.

Dode, L. et al., 2003. Dissection of the functional differences between sarco(endo)plasmic reticulum Ca2+-ATPase (SERCA) 1 and 2 isoforms and characterization of Darier disease (SERCA2) mutants by steady-state and transient kinetic analyses. The Journal of biological chemistry, 278(48), pp. 47877-89.

Fang, K. S. et al., 1998. Migration of human keratinocytes in electric fields requires growth factors and extracellular calcium. The Journal of investigative dermatology, 111(5), pp. 751-6.

Foggia, L. et al., 2006. Activity of the hSPCA1 Golgi Ca2+ pump is essential for Ca2+-mediated Ca2+ response and cell viability in Darier disease. Journal of cell science, 119(Pt 4), pp. 671-9.

Hampton, P. J. et al., 2012. Lithium regulates keratinocyte proliferation via glycogen synthase kinase 3 and NFAT2 (nuclear factor of activated T cells 2). Journal of cellular physiology, 227(4), pp. 1529-37.

Henry, J. et al., 2012. Update on the epidermal differentiation complex. Frontiers in bioscience (Landmark edition), 17(4), pp. 1517-32.

Ho, I. C. et al., 1998. A potential role for the nuclear factor of activated T cells family of transcriptional regulatory proteins in adipogenesis. Proceedings of the National Academy of Sciences of the United States of America, 95(26), pp. 15537-41.

Hoffjan, S. & Stemmler, S., 2007. On the role of the epidermal differentiation complex in ichthyosis vulgaris, atopic dermatitis and psoriasis. The British journal of dermatology, 157(3), pp. 441-9.

Hogan, P. G. et al., 2003. Transcriptional regulation by calcium, calcineurin, and NFAT. Genes & development, 17(18), pp. 2205-32.

Hohl, D. et al., 1995. The small proline-rich proteins constitute a multigene family of differentially regulated cornified cell envelope precursor proteins. The Journal of investigative dermatology, 104(6), pp. 902-9.

Hong, J. H. et al., 2010. Markers of squamous cell carcinoma in sarco/endoplasmic reticulum Ca2+ ATPase 2 heterozygote mice keratinocytes. Progress in biophysics and molecular biology, 103(1), pp. 81-7.

Hovnanian, A., 2004. Darier's disease: from dyskeratosis to endoplasmic reticulum calcium ATPase deficiency. Biochemical and biophysical research communications, 322(4), pp. 1237-44.

Ishida-Yamamoto, a et al., 1995. Altered distribution of keratinization markers in epidermolytic hyperkeratosis. Archives of dermatological research, 287(8), pp. 705-11.

Jackson, B. et al., 2005. Late cornified envelope family in differentiating epithelia—response to calcium and ultraviolet irradiation. The Journal of investigative dermatology, 124(5), pp. 1062-70.

Kanitakis, J. et al., 1987. Involucrin expression in keratinization disorders of the skin—a preliminary study. The British journal of dermatology, 117(4), pp. 479-86.

Kao, S.-C. et al., 2009. Calcineurin/NFAT signaling is required for neuregulin-regulated Schwann cell differentiation. Science (New York, N.Y), 323(5914), pp. 651-4.

Kartasova, T. & van de Putte, P., 1988. Isolation, characterization, and UV-stimulated expression of two families of genes encoding polypeptides of related structure in human epidermal keratinocytes. Molecular and cellular biology, 8(5), pp. 2195-203.

Kassar, S. et al., 2008. Immunohistological study of involucrin expression in Darier's disease skin. Journal of cutaneous pathology, 35(7), pp. 635-40.

Koizumi, H. et al., 1996. Differentiation-associated localization of small proline-rich protein in normal and diseased human skin. The British journal of dermatology, 134(4), pp. 686-92.

Leinonen, P. T. et al., 2005. Keratinocytes cultured from patients with Hailey-Hailey disease and Darier disease display distinct patterns of calcium regulation. The British journal of dermatology, 153(1), pp. 113-7.

Liu, L. H. et al., 2001. Squamous cell tumors in mice heterozygous for a null allele of Atp2a2, encoding the sarco(endo)plasmic reticulum Ca2+-ATPase isoform 2 Ca2+ pump. The Journal of biological chemistry, 276(29), pp. 26737-40.

Macián, F., López-Rodríguez, C. & Rao, a, 2001. Partners in transcription: NFAT and AP-1. Oncogene, 20(19), pp. 2476-89.

Mammucari, C. et al., 2005. Integration of Notch 1 and calcineurin/NFAT signaling pathways in keratinocyte growth and differentiation control. Developmental cell, 8(5), pp. 665-76.

Mao-Qiang, M. et al., 1997. Calcium and potassium inhibit barrier recovery after disruption, independent of the type of insult in hairless mice. Experimental dermatology, 6(1), pp. 36-40.

Marenholz, I. et al., 2001. Identification of human epidermal differentiation complex (EDC)-encoded genes by subtractive hybridization of entire YACs to a gridded keratinocyte cDNA library. Genome research, 11(3), pp. 341-55.

Mischke, D. et al., 1996. Genes encoding structural proteins of epidermal cornification and S100 calcium-binding proteins form a gene complex (“epidermal differentiation complex”) on human chromosome 1q21. The Journal of investigative dermatology, 106(5), pp. 989-92.

Miyauchi, Y. et al., 2006. Comprehensive analysis of expression and function of 51 sarco(endo)plasmic reticulum Ca2+-ATPase mutants associated with Darier disease. The Journal of biological chemistry, 281(32), pp. 22882-95.

Molin, S. et al., 2011. Deletion of the Late Corni fi ed Envelope Genes LCE3B and LCE3C May Promote Chronic Hand Eczema With Allergic Contact Dermatitis., 21(6), pp. 472-479.

Molkentin, J. D. et al., 1998. A calcineurin-dependent transcriptional pathway for cardiac hypertrophy. Cell, 93(2), pp. 215-28.

Muller, M. R. & Rao, A., 2010. NFAT, immunity and cancer: a transcription factor comes of age. Nature reviews. Immunology, 10(9), pp. 645-56.

Pani, B. et al., 2006. Up-Regulation of Transient Receptor Potential Canonical 1 (TRPC1) following Sarco (endo) plasmic Reticulum Ca 2 □ ATPase 2 Gene Silencing Promotes Cell Survival: A Potential Role for TRPC1 in Darier's Disease., 17(October), pp. 4446-4458.

Pena, J. a, Losi-Sasaki, J. L. & Gooch, J. L., 2010. Loss of calcineurin Aalpha alters keratinocyte survival and differentiation. The Journal of investigative dermatology, 130(1), pp. 135-40.

Pérez-Carmona, L. et al., Successful treatment of Darier's disease with topical pimecrolimus. European journal of dermatology: EJD, 21(2), pp. 301-2.

Reddy, S. P. M., Vuong, H. & Adiseshaiah, P., 2003. Interplay between proximal and distal promoter elements is required for squamous differentiation marker induction in the bronchial epithelium: role for ESE-1, Sp1, and AP-1 proteins. The Journal of biological chemistry, 278(24), pp. 21378-87.

Rubegni, P. et al., 2006. A case of Darier's disease successfully treated with topical tacrolimus. Journal of the European Academy of Dermatology and Venereology: JEADV, 20(1), pp. 84-7.

Sakuntabhai, a et al., 1999. Mutations in ATP2A2, encoding a Ca2+ pump, cause Darier disease. Nature genetics, 21(3), pp. 271-7.

Santini, M. P. et al., 2001. in control of p21 WAF1/CIP1 expression in keratinocyte differentiation., 98(17).

Savignac, M. et al., 2011. Darier disease : a disease model of impaired calcium homeostasis in the skin. Biochimica et biophysica acta, 1813(5), pp. 1111-7.

Savignac, M. et al., 2014. SERCA2 Dysfunction in Darier Disease Causes Endoplasmic Reticulum Stress and Impaired Cell-to-Cell Adhesion Strength: Rescue by Miglustat. The Journal of investigative dermatology, 134(7), pp. 1961-70.

Schreber, S. L., 1991. the Immunophilins Their Immunosuppressive Ligands Molecular Recognition by the Immunophilins., 11(January), pp. 283-287.

Serfling, E. et al., 2006. NFATc1 autoregulation: a crucial step for cell-fate determination. Trends in immunology, 27(10), pp. 461-9.

Shahidullah, H., Humphreys, F. & Beveridge, G. W., 1994. Darier's disease: severe eczematization successfully treated with cyclosporin. The British journal of dermatology, 131(5), pp. 713-6.

Sheng, M., McFadden, G. & Greenberg, M. E., 1990. Membrane depolarization and calcium induce c-fos transcription via phosphorylation of transcription factor CREB. Neuron, 4(4), pp. 571-82.

Siekierka, J. J. & Sigal, N. H., 1992. FK-506 and cyclosporin A: immunosuppressive mechanism of action and beyond. Current opinion in immunology, 4(5), pp. 548-52.

Stewart, L. C. & Yell, J., 2008. Vulval Darier's disease treated successfully with ciclosporin. Journal of obstetrics and gynaecology: the journal of the Institute of Obstetrics and Gynaecology, 28(1), pp. 108-9.

Sugiura, K., 2013. Unfolded protein response in keratinocytes: impact on normal and abnormal keratinization. Journal of dermatological science, 69(3), pp. 181-6.

Tabary, O. et al., 2006. Calcium-dependent regulation of NF-(kappa)B activation in cystic fibrosis airway epithelial cells. Cellular signalling, 18(5), pp. 652-60.

Vasioukhin, V. et al., 2000. Directed actin polymerization is the driving force for epithelial cell-cell adhesion. Cell, 100(2), pp. 209-19.

Wang, Q. et al., 2001 Inhibition of the phosphatidylinositol 3-kinase pathway contributes to HT29 and Caco-2 intestinal cell differentiation. Gastroenterology, 120(6), pp. 1381-1392.

Xu, L. et al., 2011. Deletion of LCE3C and LCE3B genes is associated with psoriasis in a northern Chinese population. The British journal of dermatology, 165(4), pp. 882-7.

Yang, T. T. C. et al., 2002. Phosphorylation of NFATc4 by p38 Mitogen-Activated Protein Kinases., 22(11), pp. 3892-3904.

Zhou, B. et al., 2002. Regulation of the murine Nfatc1 gene by NFATc2. The Journal of biological chemistry, 277(12), pp. 10704-11.

1. A method of treating Darier disease in a subject in need thereof comprising administering the subject with a therapeutically effective amount of a calcineurin inhibitor.

2. A method of treating Darier disease in a subject in need thereof comprising administering the subject with a therapeutically effective amount of an inhibitor of NFATC1 gene expression.
